(19) United States
(12) Patent Application Publication
(10) Pub. No.: US 2022/0241450 A1
(43) Pub. Date: Aug. 4, 2022

(54) MAL-ODOR REMOVING COMPOSITION AND RELATED METHODS THEREOF

(71) Applicant: SportSense LLC, Monticello, MN (US)

(72) Inventor: Vicki Peterson, Monticello, MN (US)

(21) Appl. No.: 17/649,279

(22) Filed: Jan. 28, 2022

Related U.S. Application Data
(60) Provisional application No. 63/143,314, filed on Jan. 29, 2021.

(51) Int. Cl. A61L 9/01

(52) U.S. Cl. CPC A61L 9/01; A61L 2101/32; A61L 2209/22

(57) ABSTRACT

Aqueous compositions of one or more organoethoxysilanes dissolved in an aqueous solution with one or more alcohols, polyvinyl alcohol and one or more optional fragrances, which can present or eliminate malodor aroma from microogranisms, methods of preparing the mal-odor removing compositions, and/or methods of using the mal-odor removing compositions. The mal-odor removing aqueous compositions preferably being in an liquid form that leaves is capable of providing clear, dried coating or film upon applied surfaces that removes odors and prevents proliferation of odor causing bacteria, fungi and viruses.

### RELATED APPLICATION

The present application claims benefit of U.S. Provisional Application No. 63/143,314, filed Jan. 29, 2021, which is hereby incorporated herein in its entirety by reference.

### TECHNICAL FIELD

The present invention relates to mal-odor removing compositions having one or more organoethoxysilane quaternary ammonium compounds dissolved in an aqueous solution with one or more alcohols, one or more water-soluble polymeric carriers and one or more optional fragrances, particularly mal-odor removing compositions preferably being in an liquid form that leaves a clear coating upon applied surfaces that removes odors and prevents proliferation of odor causing microorganisms, particularly bacteria, fungus and viruses.

### BACKGROUND

The proliferation of microorganisms (e.g., bacteria, fungi and viruses) on surfaces presents problems in numerous industries. Such industries include, for example, the medical healthcare industry, the medical device industry, construction industry, textile industry, food processing industry, food preparation industry, sporting equipment industry, the sporting apparel industry, the lumber industry, schools, law enforcement, and the textile industry, to name a few. Such problems include the transmission of infectious diseases and illness, contamination of surfaces, transmission of microorganism into food products or medical equipment, malodorous, and the like. As such, the reduction or elimination of microorganisms on various surfaces is important in a broad variety of industries and applications within those industries.

One approach to preventing the proliferation of microorganisms on surfaces is the application of antimicrobial compositions to destroy or inhibit the growth of microorganisms and especially pathogenic microorganism. Another approach is to interfere with the ability of microorganisms to survive on surfaces is to modify the surface by application of compositions, such as antimicrobial agents, prior to the attachment of microorganisms to the surface.

No matter the planned end-use of the material, important aspects of the antimicrobial agent is that it be effective, persistent, and not easily inactivated. For example in the sports industry where the sports equipment and apparel often come into contact with bodily fluids, such as sweat, it is important that the antimicrobial agent is not easily rinsed away, or otherwise inactivated, by exposure to the biological fluid. For applications in which the surface is washed or rinsed between uses, it is important that the antimicrobial agent is not easily washed or rinsed away by regular cleaning of the equipment.

It is also important that the antimicrobial agent not contain toxic or hazardous components that are undersirable for some consumers, such as the presence of methanol. Another problem to be avoided by antimicrobial agents is instability to pH changes or temperature and/or pressure changes. Still further, it is desirable to provide antimicrobial agents that are soluble in water and does not easily degrade over time when exposed to ambient conditions. It is further desirable to provide an antimicrobial agent that is cost-effective and does not take complex or time-consuming steps to produce.

Consequently, there exists a need for antimicrobial compositions (e.g., ones that include one or more organoethoxysilanes, particularly, octadecyldimethyl (3-triethoxy silylpropyl) ammonium chloride that are effective at removing malodorous aromas, as well as methods of manufacturing the same in a convenient, reliable, and cost-effective manner.

### SUMMARY OF THE INVENTION

The present invention is generally directed at an aqueous composition for eliminating or lowering malodor associated with one or more microorganisms, methods of preparing the aqueous composition, methods of using the aqueous composition by application to a surface of a substrate and methods of contacting the one or more microorganisms with the aqueous composition.

In certain aspects of the present invention, the aqueous composition is capable of effectively reducing the number of microorganisms on the substrate to eliminate or lower the malodor associated with the one or more microorganisms. In some alternative aspects, the aqueous composition inhibits the proliferation of the one or more microorganisms, such that the aqueous composition preemptively inhibits or prevents any associated malodor.

In certain aspects of the present invention, the aqueous composition is relatively inexpensive, safe, nontoxic and convenient to use. In some aspects, the aqueous composition can be applied to a surface of a substrate by at least one of spraying, dipping, brushing, wiping and rolling the aqueous composition onto at least a portion of the surface of the substrate. In some aspects, the aqueous composition can be applied to a surface by a liquid applicator comprising at least one of a spray bottle, wipe, cloth, sponge, non-woven fabric, and woven fabric.

In certain aspects, the substrate to which the aqueous composition can be applied is a topical surface of at least one of a mammal, non-woven fabric, woven fabric, natural textile, synthetic textile, leather, organic particulate, inorganic particulate, fiber, agglomerate, foam, film, cellulosic material, metal, plastic, natural rubber, synthetic rubber, glass, paint, stain, adhesive, stone, grout, fiberglass, medical device, clothing apparel, sporting equipment, wood, concrete, construction product, building product, and activated carbon.

In some aspects, the aqueous composition can be applied to a fabric substrate, including at least one of polyester fabric, synthetic polyester fabric, non-engineered polyester fabric, performance apparel, moisture wicking performance fabric, delicate moisture wicking performance fabric, and moisture wicking performance apparel.

In some aspects, the aqueous composition can be applied to equipment, athletic gear, athletic apparel and/or athletic footwear.

In some aspects, the aqueous composition is applied to a surface for eliminating or lowering malodor associated with one or more microorganisms selected from the group consisting of bacterium, a virus, fungus, mold, slime mold, algae and yeast. In some aspects, the aqueous composition is an antimicrobial, such that the aqueous composition can effectively kill or inhibit microorganism growth or the proliferation of microorganisms on a substrate.

In some aspects of the present invention, the aqueous composition includes (a) one or more organoethoxysilane quaternary ammonium compounds, (b) one or more water-soluble polymers, (c) one or more alcohols, (d) water, and (e) one or more optional fragrances.

In certain preferred aspects, the aqueous composition is substantially devoid of any organomethoxysilane quaternary ammonium compounds. In other certain preferred aspects, the the aqueous composition and the starting components to manufacture the aqueous composition are substantially devoid of methanol.

In certain aspects, the one or more organoethoxysilane quaternary ammonium compounds is present in an amount between about 0.5 wt-% and about 4.0 wt-%, preferably between about 0.6 wt-% and about 3.5 wt-%, preferably between about 0.7 wt-% and about 3.0 wt-%, preferably between about 0.75 wt-% and about 2.5 wt-%, preferably between about 0.8 wt-% and about 2.0 wt-%, preferably between about 0.85 wt-% and about 1.5 wt-%, more preferably between about 0.9 wt-% and about 1.25 wt-%.

In certain aspects, the one or more organoethoxysilane quaternary ammonium compounds is of general formula (I):

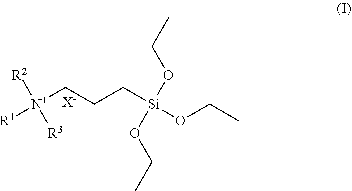

wherein R¹ is an alkyl group having from 12 to 24 carbon atoms, preferably 14-18 carbon atoms, most preferably 16 carbon atoms; R² is a 3-triethoxysilylpropyl group or a lower alkyl group having from 1 to 6 carbon atoms, preferably 1 to 4 carbon atoms, more preferably 1 to 2 carbon atoms; R³ is a hydrogen atom or a lower group having from 1 to 6 carbon atoms, preferably 1 to 4 carbon atoms, more preferably 1 to 2 carbon atoms; and X represents a halogen ion, preferably the hydrogen ion being a chlorine ion.

In certain aspects, the one or more organoethoxysilane quaternary ammonium compounds comprises octadecyldimethyl (3-triethoxy silylpropyl) ammonium chloride, octadecylbis(triethoxy silylpropyl) ammonium chloride, or combinations thereof. In certain preferred aspects, the one or more organoethoxysilane quaternary ammonium compounds comprises octadecyldimethyl (3-triethoxy silylpropyl) ammonium chloride. In still other certain preferred aspects, the the one or more organoethoxysilane quaternary ammonium compounds consists of octadecyldimethyl (3-triethoxy silylpropyl) ammonium chloride. In some aspects, the aqueous composition is substantially devoid of any organomethoxysilane quaternary ammonium compounds, including octadecyldimethyl(trimethoxysilylpropyl) ammonium chloride.

In certain aspects, the one or more organoethoxysilane quaternary ammonium compounds, such as octadecyldimethyl (3-triethoxy silylpropyl) ammonium chloride, is provided in an ethanol solution, such that the aqueous composition comprises ethanol. In some aspects, the one or more organoethoxysilane quaternary ammonium compounds (grams) is provided in an ethanol solution such that the amount of the one or more organoethoxysilane quaternary ammonium compounds (grams) is present in the ethanol solution (100 mL) in an amount between about 20% and about 85%, preferably between about 40% and about 80%, more preferably between about 50% and about 75%, most preferably about 72%.

In certain aspects, the aqueous composition comprises ethanol in an amount between about 0.25 wt-% and about 15.0 wt-%, preferably between about 0.26 wt-% and about 12 wt-%, preferably between about 0.27 wt-% and about 10.0 wt-%, preferably between about 0.28 wt-% and about 8.0 wt-%, preferably between about 0.30 wt-% and about 6.0 wt-%, preferably between about 0.32 wt-% and about 4.0 wt-%, preferably between about 0.34 wt-% and about 2.0 wt-%, most preferably between about 0.35 wt-% and about 1.0 wt-%.

In certain aspects, the one or more water-soluble polymers is present in an amount between about 0.1 wt-% and about 10.0 wt-%, preferably between about 0.15 wt-% and about 5.0 wt-%, preferably between about 0.2 wt-% and about 2.5 wt-%, preferably between about 0.25 wt-% and about 2.0 wt-%, preferably between about 0.3 wt-% and about 1.5 wt-%, preferably between about 0.35 wt-% and about 1.0 wt-%, more preferably between about 0.4 wt-% and about 0.75 wt-%.

In certain aspects, the one or more water-soluble polymers has a solubility in water of at least about 0.05 g/mL at 80° C. and 1 atm, at least about 0.075 g/mL at 80° C. and 1 atm, or at least about 0.1 g/mL at 80° C. and 1 atm.

In certain aspects, the one or more water-soluble polymers comprises polyvinyl alcohol, polyvinyl acetate, modified starch, functionalized starch, polyethylene glycol, ethylene vinyl alcohol, cellulose, cellulose acetate, chitosan, or combinations thereof. In certain preferred aspects, the one or more water-soluble polymers comprises polyvinyl alcohol. In certain other preferred aspects, the one or more water-soluble polymers consists of polyvinyl alcohol.

In certain aspects, the one or more alcohols is present in an amount between about 0.5 wt-% and about 25.0 wt-%, preferably between about 2.0 wt-% and about 20.0 wt-%, preferably between about 4.0 wt-% and about 17.5 wt-%, preferably between about 5.0 wt-% and about 15.0 wt-%, preferably between about 6.0 wt-% and about 12.5 wt-%, preferably between about 7.0 wt-% and about 11.0 wt-%, more preferably between about 8.0 wt-% and about 10.5 wt-%.

In certain aspects, the one or more alcohols is isopropanol, ethanol or combinations thereof. In certain aspects, the one or more alcohols comprises isopropanol. In certain preferred aspects, the one or more alcohols comprises isopropanol and ethanol is present in the aqueous composition by providing the one or more organoethoxysilane quaternary ammonium compounds dissolved in ethanol.

In certain aspects, water is present in an amount between about 20 wt-% and about 98 wt-%, preferably between about 50 wt-% and about 95 wt-%, preferably between about 75 wt-% and about 92.5 wt-%, preferably between about 80 wt-% and about 90 wt-%, preferably between about 85 wt-% and about 89.5 wt-%, more preferably between about 87.5 wt-% and about 89 wt-%. In certain preferred aspects, the water is deionized water.

In certain aspects, the one or more optional fragrances in an amount between about 0 wt-% and about 5 wt-%, preferably between about 0.02 wt-% and about 2.5 wt-%, preferably between about 0.025 wt-% and about 2.0 wt-%, preferably between about 0.05 wt-% and about 1.0 wt-%, preferably between about 0.075 wt-% and about 0.75 wt-%, more preferably between about 0.08 wt-% and about 0.5 wt-%.

In certain aspects, the aqueous composition and the components of the aqueous composition are substantially devoid of an anti-foaming agent, preferably devoid of a silicon anti-foaming agent.

In certain preferred aspects of the present invention, the aqueous composition comprises (a) about 0.025 wt-% to about 2.5 wt-% of octadecyldimethyl (3-triethoxysilylpropyl) ammonium chloride, (b) about 0.25 wt-% to about 2.0 wt-% of polyvinyl alcohol, (C) about 0.25 wt-% to about 2.0 wt-% of ethanol, (d) about 0.25 wt-% to about 15.0 wt-% isopropanol, (e) about 75 wt-% to about 95 wt-% water, and (f) about 0 wt-% to about 1.0 wt-% of the one or more optional fragrances.

In certain aspects, the aqueous composition is a clear liquid such that a precipitate in the aqueous composition is not visible to an unaided eye of an ordinary observer.

In certain preferred aspects, the aqueous composition is manufactured by dissolving into water the one or more organoethoxysilane quaternary ammonium compounds, the one or more water-soluble polymers, the one or more alcohols, and the one or more optional fragrances, to provide the aqueous composition.

In certain aspects, the one or more polymers are added to water and heated to dissolve the one or more polymers into water to provide an aqueous polymeric solution. In certain aspects, the one or more polymers and water are heated to a temperature of about 90° F. to about 200° F., more preferably about 100° F. to about 190° F., more preferably about 115° F. to about 180° F., more preferably about 125° F. to about 170° F., more preferably about 140° F. to about 160° F., even more preferably about 145° F. to about 155° F. In certain preferred aspects, the one or more polymers are added to the water and heated to a temperature below about 175° F., more preferably below about 170° F., more preferably below about 165° F., more preferably about 160° F., and even more preferably about 155° F.

In certain preferred aspects, the aqueous polymeric solution is cooled to a temperature below about 100° F., more preferably below about 90° F., more preferably below about 80° F., more preferably below about 75° F., more preferably about 70° F., even more preferably about ambient room temperature, before the one or more alcohols are mixed into the aqueous polymeric solution.

In certain preferred aspects, the aqueous polymeric solution is cooled to a temperature below about 100° F., more preferably below about 90° F., more preferably below about 80° F., more preferably below about 75° F., more preferably about 70° F., even more preferably about ambient room temperature, before the one or more organoethoxysilane quaternary ammonium compounds are mixed into the aqueous polymeric solution.

In certain preferred aspects, the one or more organoethoxysilane quaternary ammonium compounds, the one or more alcohols and the one or more optional fragrances are mixed into the aqueous polymeric solution after the aqueous polymeric solution is cooled to a temperature below about 100° F., more preferably below about 90° F., more preferably below about 80° F., more preferably below about 75° F., more preferably about 70° F., even more preferably about ambient room temperature. In certain aspects, the one or more organoethoxysilane quaternary ammonium compounds are mixed into the aqueous polymeric solution after the one or more alcohols are mixed into the aqueous polymeric solution. In certain other aspects, the one or more organoethoxysilane quaternary ammonium compounds are mixed into the aqueous polymeric solution before the one or more alcohols are mixed into the aqueous polymeric solution.

The above summary is not intended to describe each illustrated embodiment or every implementation of the subject matter hereof. The detailed description and example that follow more particularly exemplify various embodiments.

### DETAILED DESCRIPTION OF THE INVENTION

The present invention relates to an aqueous composition having one or more organoethoxysilane quaternary ammonium compounds, one or more water-soluble polymers, one or more alcohols, and one or more optional fragrances in water. The aqueous composition is preferably manufactured into a substantially clear liquid that can be used for eliminating or lowering malodor associated with one or more microorganisms. In some contemplated aspects, the aqueous composition will undergo an evaporation process of the one or more alcohols and water, such that the residue will comprise the one or more organoethoxysilane quaternary ammonium compounds, the one or more water-soluble polymers, and the one or more optional fragrances. In some aspects, the evaporation process will occur after the aqueous composition is applied to a desired surface of a substrate. In still other aspects, it is contemplated that the aqueous composition may undergo an evaporation process to provide a dry, solid (e.g., powdered) form that can be applied to a desired surface of a substrate, or such that the dry, solid form can be dissolved in a desired liquid carrier by the end-user. In certain aspects, the dry, solid form of the composition is water-soluble, such that the composition has a solubility in water of at least about 0.05 g/mL at 80° C. and 1 atm at least about 0075 g/mL at 80° C. and 1 atm, or at least about 0.1 g/mL at 80° C. and 1 atm.

The one or more organoethoxysilane quaternary ammonium compounds are employed to effectively reduce the number of microorganisms on the substrate to eliminate or lower the malodor associated with the one or more microorganisms. In some aspects, the one or more organoethoxysilane quaternary ammonium compounds are chosen to inhibit the proliferation of the one or more microorganisms, such that the one or more organoethoxysilane quaternary ammonium compounds kills or acts as an antimicrobial agent.

Preferred organoethoxysilane quaternary ammonium compounds are contemplated to have general formula (I):

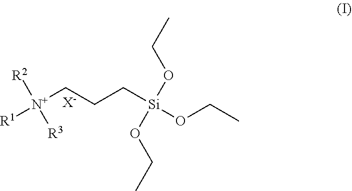

wherein R¹ is an alkyl group having from 12 to 24 carbon atoms, preferably 14-18 carbon atoms, most preferably 16 carbon atoms; R² is a 3-triethoxysilylpropyl group or a lower alkyl group having from 1 to 6 carbon atoms, preferably 1 to 4 carbon atoms, more preferably 1 to 2 carbon atoms; R³ is a hydrogen atom or a lower group having from 1 to 6 carbon atoms, preferably 1 to 4 carbon atoms, more preferably 1 to 2 carbon atoms; and X represents a halogen ion, preferably the hydrogen ion being a chlorine ion (Cl—).

A preferred organoethoxysilane quaternary ammonium compound used in the present invention includes octadecyldimethyl (3-triethoxysilylpropyl) ammonium chloride having formula (II):

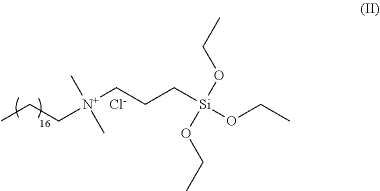

Octadecyldimethyl (3-triethoxysilylpropyl) ammonium chloride is commercially available as a liquid, 72% in ethanol from Gelest™ (Product Code: SIO6619.2, CAS No. 62117-57-1) having a molecular mass of 538.37 g/mol, boiling point of 76° C., relative density of 0.86-0.89 and solubility in water.

It is contemplated that another suitable organoethoxysilane quaternary ammonium compound that can be used in the present invention includes octadecylbis(triethoxysilylpropyl) ammonium chloride having formula (III):

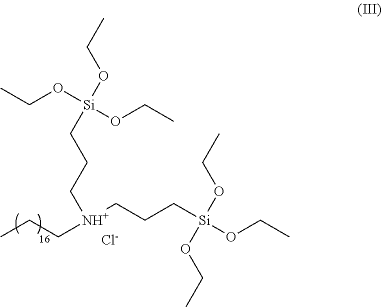

In certain aspects, the one or more organoethoxysilane quaternary ammonium compounds comprises octadecyldimethyl (3-triethoxy silylpropyl) ammonium chloride, octadecylbis(triethoxy silylpropyl) ammonium chloride, or combinations thereof. In certain preferred aspects, the one or more organoethoxysilane quaternary ammonium compounds comprises octadecyldimethyl (3-triethoxy silylpropyl) ammonium chloride. In still other certain preferred aspects, the one or more organoethoxysilane quaternary ammonium compounds consists of octadecyldimethyl (3-triethoxy silylpropyl) ammonium chloride. In some aspects, the aqueous composition is substantially devoid of any organomethoxysilane quaternary ammonium compounds, including octadecyldimethyl(trimethoxysilylpropyl) ammonium chloride.

The one or more organoethoxysilane quaternary ammonium compounds can be employed in any suitable amount, provided that the amount is effective in inhibiting the growth of the desired microorganism. For example, the one or more organoethoxysilane quaternary ammonium compounds can be employed in an amount up to about 10 wt-% of the aqueous composition, preferably in an amount between about 0.5 wt-% and about 4.0 wt-%, preferably between about 0.6 wt-% and about 3.5 wt-%, preferably between about 0.7 wt-% and about 3.0 wt-%, preferably between about 0.75 wt-% and about 2.5 wt-%, preferably between about 0.8 wt-% and about 2.0 wt-%, preferably between about 0.85 wt-% and about 1.5 wt-%, more preferably between about 0.9 wt-% and about 1.25 wt-%, of the aqueous composition.

In some aspects, the one or more organoethoxysilane quaternary ammonium compounds will be provided during the manufacturing process in an alcohol solution. For example, as provided above, octadecyldimethyl (3-triethoxysilylpropyl) ammonium chloride can be provided as a liquid, 72% in ethanol. In some preferred aspects, the one or more organoethoxysilane quaternary ammonium compounds during the manufacturing process is provided in an ethanol solution, such that the aqueous composition contains ethanol. For example, the one or more organoethoxysilane quaternary ammonium compounds (grams) can be provided in an ethanol solution (100 mL) in an amount between about 20% and about 85%, preferably between about 40% and about 80%, more preferably between about 50% and about 75%, most preferably about 72%.

In other certain preferred aspects, the one or more organoethoxysilane quaternary ammonium. compounds is provided in a form devoid of methanol, such that the aqueous composition is substantially devoid of methanol. In some alternative aspects where the one or more organoethoxysilane quaternary ammonium compounds are provided in an methanol solution, the methanol is removed during the manufacturing process, such that the aqueous composition is substantially devoid of methanol. In preferred aspects, the one or more organoethoxysilane quaternary ammonium compounds are provided as a solid or in a liquid solution that does not contain methanol, such that both the starting components to manufacture the aqueous composition and the resulting aqueous composition are substantially devoid of methanol (less than 0.001 wt-% methanol).

The aqueous composition described herein will include one or more water-soluble polymers. Water solubility will help provide a suitable aqueous composition On which the one or more organoethoxysilane quaternary ammonium compounds is complexed to the one or more water-soluble polymers) to be effectively delivered to a desired surface of a substrate. In certain aspects, the one or more water-soluble polymers has a solubility in water of at least about 0.05 g/mL at 80° C. and 1 atm, at least about 0.075 g/mL at 80° C. and 1 atm, or at least about 0.1 g/mL at 80° C. and 1 atm.

While it is contemplated that any suitable water-soluble polymer can be employed, preferred water-soluble polymers include polyvinyl alcohol, polyvinyl acetate, modified starch, functionalized starch, polyethylene glycol, ethylene vinyl alcohol, cellulose, cellulose acetate, chitosan, or combinations thereof. In certain preferred aspects, the one or more water-soluble polymers comprises polyvinyl alcohol. In certain other preferred aspects, the one or more water-soluble polymers consists of polyvinyl alcohol.

The one or more water-soluble polymers can be employed in any suitable amount, provided that the one or more water-soluble polymers effectively dissolve into solution and remain in solution. upon the aqueous composition being stored at ambient temperature. For example, the one or more water-soluble polymers can be employed in an amount up to about 10 wt-% of the aqueous composition, preferably in an amount between about 0.1 wt-% and about 10.0 wt-%, preferably between about 0.15 wt-% and about 5.0 wt-%, preferably between about 0.2 wt-% and about 2.5 wt-%, preferably between about 0.25 wt-% and about 2.0 wt-%, preferably between about 0.3 wt-% and about 1.5 wt-%, preferably between about 0.35 wt-% and about 1.0 wt-%, more preferably between about 0.4 wt-% and about 0.75 wt-%, of the aqueous composition.

It is contemplated that the one or more organoethoxysilane quaternary ammonium compounds can be complexed with the one or more water-soluble polymers. For example, it is contemplated that at least about 20 mol. % of the one or more organoethoxysilane quaternary ammonium compounds is complexed with the one or more water-soluble polymers, at least about 40 mol. % of the one or more organoethoxysilane quaternary ammonium compounds is complexed with the one or more water-soluble polymers, at least about 60 mol. % of the one or more organoethoxysilane quaternary ammonium compounds is complexed with the one or more water-soluble polymers, at least about 80 mol. % of the one or more organoethoxysilane quaternary ammonium compounds is complexed with the one or more water-soluble polymers, at least about 90 mol. % of the one or more organoethoxysilane quaternary ammonium compounds is complexed with the one or more water-soluble polymers, or at least about 95 mol. % of the one or more organoethoxysilane quaternary ammonium compounds is complexed with the one or more water-soluble polymers.

The weight ratio of the one or more organoethoxysilane quaternary ammonium compounds to the water-soluble polymer can be about 1:10 to about 10:1, preferably about 1:5 to about 5:1, more preferably about 1:3 to about 3:1, more preferably about 1:2 to about 2:1, more preferably about 1:1 to about 3:1.

The aqueous composition described herein will include one or more alcohols, which may serve as both a liquid carrier and an antimicrobial. While it is contemplated that any suitable alcohol can be employed, preferred alcohols include isopropanol, ethanol or combinations thereof. In certain preferred aspects, the one or more alcohols comprises isopropanol. In still other certain preferred aspects, the one or more alcohols comprises isopropanol, such that any ethanol present in the aqueous composition is provided from the source of the one or more organoethoxysilane quaternary ammonium compounds being provided in an ethanol solution.

The one or more alcohols can be employed in any suitable amount, provided that the one or more alcohols are effectively miscible in water and maintain the one or more organoethoxy silane quaternary ammonium compounds in solution when the aqueous composition is stored at ambient temperature. For example, the one or more alcohols can be employed in an amount up to about 25 wt-%, preferably in an amount between about 0.5 wt-% and about 25.0 wt-%, preferably between about 2.0 wt-% and about 20.0 wt-%, preferably between about 4.0 wt-% and about 17.5 wt-%, preferably between about 5.0 wt-% and about 15.0 wt-%, preferably between about 6.0 wt-% and about 12.5 wt-%, preferably between about 7.0 wt-% and about 11.0 wt-%, more preferably between about 8.0 wt-% and about 10.5 wt-%.

The water in the aqueous composition can be employed in any suitable amount, provided that the other components effectively remain dissolved in solution and provided that the other components are present in an amount to be effective in inhibiting the growth of the desired microorganism. For example, the water can be employed in an amount up to about 98 wt-%, preferably in an amount between about 20 wt-% and about 98 wt-%, preferably between about 50 wt-% and about 95 wt-%, preferably between about 75 wt-% and about 92.5 wt-%, preferably between about 80 wt-% and about 90 wt-%, preferably between about 85 wt-% and about 89.5 wt-%, more preferably between about 87.5 wt-% and about 89 wt-%, of the aqueous composition. In certain preferred aspects, the water is deionized water.

While the aqueous composition can be relatively odorless, which may be desirable to some consumers, the aqueous composition may have one or more optional fragrances. The one or more optional fragrances can be employed in any suitable amount that does not provide an offensive or unpleasant odor or otherwise discolor or stain substrates, such as clothing apparel or other fabric material. For example, the one or more fragrances can be employed in an amount up to about 5 wt-%, preferably between about 0 wt-% and about 5 wt-%, preferably between about 0.02 wt-% and about 2.5 wt-%, preferably between about 0.025 wt-% and about 2.0 wt-%, preferably between about 0.05 wt-% and about 1.0 wt-%, preferably between about 0.075 wt-% and about 0.75 wt-%, more preferably between about 0.08 wt-% and about 0.5 wt-%.

In certain aspects, the aqueous composition and the components of the aqueous composition are substantially devoid of an anti-foaming agent, preferably devoid of a silicon anti-foaming agent.

In certain preferred aspects of the present invention, the aqueous composition comprises (a) about 0.025 wt-% to about 2.5 wt-% of octadecyldimethyl (3-triethoxysilylpropyl) ammonium chloride, (b) about 0.25 wt-% to about 2.0 wt-% of polyvinyl alcohol, (c) about 0.25 wt-% to about 2.0 wt-% of ethanol, (d) about 0.25 wt-% to about 4.0 wt-% isopropanol, (e) about 75 wt-% to about 95 wt-% water, and (f) about 0 wt-% to about 1.0 wt-% of the one or more optional fragrances.

The present invention also provides for methods of preparing the aqueous composition of the present invention. The methods of manufacture can include dissolving into water the one or more organoethoxysilane quaternary ammonium compounds, the one or more water-soluble polymers, the one or more alcohols, and the one or more optional fragrances, to provide the aqueous composition.

In certain preferred aspects, the one or more polymers are added to water and heated to dissolve the one or more polymers into water to provide an aqueous polymeric solution. While the mixture of the one or more polymers in water can be heated to a temperature and stirred to dissolve the one or more polymers into the water to provide an aqueous polymeric solution, the one or more polymers and water are preferably heated to a temperature of about 90° F. to about 200° F., more preferably about 100° F. to about 190° F., more preferably about 115° F. to about 180° F., more preferably about 125° F. to about 170° F., more preferably about 140° F. to about 160° F., even more preferably about 145° F. to about 155° F. In certain preferred aspects, the one or more polymers are added to the water and heated to a temperature below about 175° F., more preferably below about 170° F., more preferably below about 165° F., more preferably about 160° F., and even more preferably about 155° F., while being stirred to dissolve the one or more polymers into the water and provide the aqueous polymeric solution.

After the one or more polymers are dissolved into the water to provide the aqueous polymeric solution, the aqueous polymeric solution is then preferably cooled to a temperature below about 100° F., more preferably below about 90° F., more preferably below about 80° F., more preferably below about 75° F., more preferably about 70° F., even more preferably about ambient room temperature, before the one or more alcohols and the one or more organoethoxysilane quaternary ammonium compounds are mixed into the aqueous polymeric solution.

In certain preferred aspects, the one or more organoethoxysilane quaternary ammonium compounds, the one or more alcohols and the one or more optional fragrances are mixed into the aqueous polymeric solution after the aqueous polymeric solution is cooled to a temperature below about 100° F., more preferably below about 90° F., more preferably below about 80° F., more preferably below about 75° F., more preferably about 70° F., even more preferably about ambient room temperature. In certain aspects, the one or more organoethoxysilane quaternary ammonium compounds are mixed into the aqueous polymeric solution after the one or more alcohols are mixed into the aqueous polymeric solution. In certain other aspects, the one or more organoethoxysilane quaternary ammonium compounds are mixed into the aqueous polymeric solution before the one or more alcohols are mixed into the aqueous polymeric solution. The one or more optional fragrances can also be mixed into the solution after the aqueous polymeric solution is cooled.

The resulting aqueous composition is a clear liquid such that a precipitate in the aqueous composition is not visible to an unaided eye of an ordinary observer. The aqueous composition preferably remains a clear liquid while being stored at ambient temperature.

Without wishing to be bound by theory, when applied to a substrate, it is believed that the PVA provides a clear coating, and the one or more organoethoxysilane quaternary ammonium compounds protects this coating. The coating is capable of removing odors and prevents odor causing bacteria growth.

The present invention also provides for methods of applying the aqueous composition to a surface of a substrate or otherwise contacting the one or more microorganisms with the aqueous composition. The aqueous composition can be configured for use in a wide-variety of compositions. For example, the composition can be configured for use in a variety of manufacturing processes, such as a liquid solution in the manufacture of plastic, rubber, fabric, or textiles. The aqueous composition can also be configured for use in an athletic equipment composition, an athletic gear composition, an athletic apparel composition, or an athletic footwear composition. More specifically, because the composition can readily be configured for use in a sprayable composition, the aqueous antimicrobial composition can be configured for use in a sprayable athletic equipment composition, a sprayable athletic gear composition, a sprayable athletic apparel composition, or a sprayable athletic footwear composition.

Exemplary applications of the aqueous composition to a surface of a substrate includes at least one of spraying, dipping, brushing, wiping and rolling the aqueous composition onto at least a portion of the surface of the substrate. In some aspects, the aqueous composition can be applied to a surface by a liquid applicator comprising at least one of a spray bottle, wipe, cloth, sponge, non-woven fabric, and woven fabric.

The aqueous composition of the present invention can be applied in liquid form to a substrate, which will result in the liquid components (e.g., alcohol and water) evaporating and leaving the one or more or more organoethoxysilane quaternary ammonium compounds and one or more water-soluble polymers remaining on the substrate as a dried film or coating on the substrate. In other alternative embodiments, the one or more alcohols and water are removed from the aqueous composition to form a solid, dry form, which can be applied to a substrate.

Exemplary substrates to which the aqueous composition can be applied includes a topical surface of at least one of a mammal, non-woven fabric, woven fabric, natural textile, synthetic textile, organic particulate, inorganic particulate, fiber, agglomerate, foam, film, cellulosic material, metal, plastic, natural rubber, synthetic rubber, glass, paint, stain, adhesive, stone, grout, fiberglass, medical device, clothing apparel, sporting equipment, wood, concrete, construction product, building product, and activated carbon.

Exemplary fabric substrate to which the aqueous composition can be applied includes at least one of polyester fabric, synthetic polyester fabric, non-engineered polyester fabric, performance apparel, moisture wicking performance fabric, delicate moisture wicking performance fabric, and moisture wicking performance apparel.

The aqueous composition preferably can be applied to equipment, athletic gear, athletic apparel and/or athletic footwear.

In some aspects, the aqueous composition, or the dried film or coating resulting from application of the aqueous composition to a substrate, can eliminate or lower malodor associated with one or more microorganisms selected from the group consisting of bacterium, a virus, fungus, mold, slime mold, algae and yeast. It is also contemplated that the aqueous composition, or the dried film or coating resulting from application of the aqueous composition to the substrate, can provide antimicrobial properties to the substrate, such that the aqueous composition can effectively kill or inhibit microorganism growth or the proliferation of microorganisms on the substrate.

When the aqueous antimicrobial compositions are applied to a substrate to provide a coating or film on the substrate, the resulting dried film or coating can remain relatively stable and retain the antimicrobial properties over extended periods of time. For example, the coating or film can remain stable and retain the antimicrobial properties for at least about 1 year. In specific embodiments, the coating or film can remain stable and retain the antimicrobial properties for a contemplated period of time of about 1 to about 5 years. In additional specific embodiments, the dried coating or film resulting from the aqueous composition can remain stable and retain the antimicrobial properties for a contemplated period of time of about 1 to about 10 years.

### EXAMPLE

A representative example of the aqueous composition of the present invention can be formulated from the components provided in Table 1.

TABLE 1
Representative Exemplary Aqueous Composition Formulation.

| Component | Weight- % |
|---|---|
| Deionized Water | 80-96 |
| Polyvinyl Alcohol | 0.1-2.0 |
| Isopropanol | 5.0-15.0 |
| Octadecyldimethyl(3-triethoxy silylpropyl) | 0.025-4.0 |
| ammonium chloride, 72% in ethanol | |
| Fragrance (optional) | 0-1.0 |

A preferred representative example of the aqueous composition of the present invention can be formulated from the components provided in Table 2.

TABLE 2
Representative Preferred Aqueous Composition Formulation.

| Component | Weight- % |
|---|---|
| Deionized Water | 83-93 |
| Polyvinyl Alcohol | 0.45-0.55 |
| Isopropanol | 8.0-12.0 |
| Octadecyldimethyl(3-triethoxy silylpropyl) | 0.75-1.25 |
| ammonium chloride, 72% in ethanol | |
| Fragrance (optional) | 0-0.25 |

The aqueous composition of the preceding formulations can be prepared by stirring polyvinyl alcohol powder into deionized water at room temperature and then heating to a temperature between about 140° F. and about 160° F. until the polyvinyl alcohol has visually dissolved into the water to provide an aqueous polymeric solution. The aqueous polymeric solution is then cooled to a temperature below 140° F., preferably below about 100° F., and more preferably to ambient room temperature before adding the octadecyldimethyl(3-triethoxy silylpropyl) ammonium chloride, 72% in ethanol and/or the isopropanol and stirring to dissolve the components into solution. While the the octadecyldimethyl(3-triethoxy silylpropyl) ammonium chloride, 72% in ethanol and the isopropanol can be added to the aqueous polymeric solution at the same time, it is preferred that the isopropanol is mixed into the aqueous polymeric solution before the octadecyldimethyl(3-triethoxy silylpropyl) ammonium chloride, 72% in ethanol, is added and mixed to effectuate dissolving the components into solution. The optional fragrances are preferably the last component mixed into the solution. The resulting aqueous compositions have a clear liquid appearance, such that a precipitate in the aqueous composition is not visible to an unaided eye of an ordinary observer.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments.

1. A method of eliminating or lowering malodor associated with one or more microorganisms, the method comprising:
contacting the one or more microorganisms with an aqueous composition, wherein the aqueous composition comprises one or more organoethoxysilane quaternary ammonium compounds present in an amount between about 0.5 wt-% and about 4.0 wt-%, one or more water-soluble polymers present in an amount between about 0.1 wt-% and about 10.0 wt-%, one or more alcohols present in an amount between about 2.0 wt-% and about 20.0 wt-%, water present in an amount between about 75 wt-% and about 92.5 wt-%, and one or more optional fragrances.

2. The method of claim 1, wherein the one or more organoethoxysilane quaternary ammonium compounds is of general formula (I): 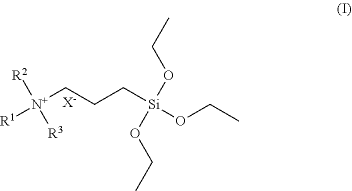
wherein R¹ is an alkyl group having 14-18 carbon atoms, R² is a 3-triethoxysilylpropyl group or a lower alkyl group having from 1 to 4 carbon atoms, R³ is a hydrogen atom or a lower group having from 1 to 4 carbon atoms, and X is a halogen ion.

3. The method of claim 3, wherein the amount of the one or more organoethoxysilane quaternary ammonium compounds is between about 0.85 wt-% and about 1.5 wt.

4. The method of claim 1, wherein the one or more organoethoxysilane quaternary ammonium compounds comprises octadecyldimethyl (3-triethoxy silylpropyl) ammonium chloride, octadecylbis(triethoxy silylpropyl) ammonium chloride, or a combination thereof.

5. The method of claim 1, wherein the one or more organoethoxysilane quaternary ammonium compounds comprises octadecyldimethyl (3-triethoxy silylpropyl) ammonium chloride.

6. The method of claim 1, wherein the aqueous composition is substantially devoid of any organomethoxysilane quaternary ammonium compounds.

7. The method of claim 1, the aqueous composition is substantially devoid of methanol.

8. The method of claim 1, wherein the one or more water-soluble polymers comprises polyvinyl alcohol, polyvinyl acetate, modified starch, functionalized starch, polyethylene glycol, ethylene vinyl alcohol, cellulose, cellulose acetate, chitosan, or a combination thereof.

9. The method of claim 8, wherein the amount of the one or more water-soluble polymers is between about 0.35 wt-% and about 1.0 wt-%.

10. The method of claim 9, wherein the one or more water-soluble polymers has a solubility in water of at least about 0.075 g/mL at 80° C. and 1 atm.

11. The method of claim 10, wherein the one or more water-soluble polymers comprises polyvinyl alcohol.

12. The method of claim 1, wherein the one or more alcohols comprises isopropanol, ethanol, or combinations thereof.

13. The method of claim 12, wherein the amount of the one or more alcohols is between about 5.0 wt-% and about 15.0 wt-%.

14. The method of claim 12, wherein the one or more alcohols comprises ethanol in an amount between about 0.30 wt-% and about 6.0 wt-%.

15. The method of claim 12, wherein the one or more alcohols comprises isopropanol and ethanol, and wherein the ethanol is provided from the starting material of a solution of the one or more organoethoxysilane quaternary ammonium compounds dissolved in ethanol.

16. The method of claim 1, wherein water is deionized water, and wherein the amount of the deionized water is between about 85 wt-% and about 89.5 wt-%.

17. The method of claim 1, wherein the one or more optional fragrances is present in an amount between about about 0.05 wt-% and about 1.0 wt-%.

18. The method of claim 1, wherein aqueous composition is substantially devoid of a silicon anti-foaming agent.

19. The method of claim 1, wherein the contacting step comprises applying the aqueous composition onto a surface of a substrate by at least one of spraying, dipping, brushing, wiping and rolling the aqueous composition onto at least a portion of the surface of the substrate.

20. An aqueous composition for eliminating or lowering malodor associated with one or more microorganisms, the aqueous composition comprising:
one or more organoethoxysilane quaternary ammonium compounds present in an amount between about 0.025 wt-% to about 2.5 wt-%;
one or more water-soluble polymers present in an amount between about 0.25 wt-% to about 2.0 wt-%;
two or more alcohols comprising ethanol in an amount between about 0.25 wt-% to about 2.0 wt-% and isopropanol in an amount between about 0.25 wt-% to about 15.0 wt-%;
water in an amount between about 75 wt-% to about 95 wt-%; and
one or more optional fragrances in amount between about 0 wt-% to about 1.0 wt-%.
